(19) United States
(12) Patent Application Publication
(10) Pub. No.: US 2022/0379122 A1
(43) Pub. Date: Dec. 1, 2022

(54) Manufacturing Method for an Implantable Medical Device

(71) Applicant: BIOTRONIK SE & Co. KG, Berlin (DE)

(72) Inventor: Marcel Starke, Eichwalde (DE)

(73) Assignee: BIOTRONIK SE & Co. KG, Berlin (DE)

(21) Appl. No.: 17/775,448

(22) PCT Filed: Dec. 9, 2020
(86) PCT No.: PCT/EP2020/085165

§ 371 Date: May 9, 2022

(30) Foreign Application Priority Data
Jan. 9, 2020 (EP) 20150884.3

(51) Int. Cl. A61N 1/375; A61N 1/378

(52) U.S. Cl. CPC A61N 1/3756; A61N 1/378; A61B 5/686

(57) ABSTRACT

A medical device and a manufacturing method for such medical device having an assembly comprising:
an elongated solid housing with an outer surface and a maximum outer diameter,
at least one electrical contact area at the outer surface of the housing, and
a processor encapsulated within the housing,
   wherein the method comprises the following steps:
providing the assembly and a tube consisting of a plastic and electrically insulating material, wherein an inner diameter of the tube is greater than the maximum outer diameter (108) of the assembly,
accommodating the assembly within the tube such that at least one electrical contact area of the assembly is not covered, and
applying a shrinking step to the tube such that the shrunken tube is firmly attached to the outer surface of the housing.

The manufacturing method is cheaper and less time consuming than state-of-the-art methods, and also better suitable for automation.

### CROSS-REFERENCE TO RELATED APPLICATIONS

This application is the United States National Phase under 35 U.S.C. § 371 of PCT International Patent Application No. PCT/EP2020/085165, filed on Dec. 9, 2020, which claims the benefit of European Patent Application No. 20150884.3, filed on Jan. 9, 2020, the disclosures of which are hereby incorporated by reference herein in their entireties.

### TECHNICAL FIELD

The present invention relates to a manufacturing method for an implantable medical device and a respective medical device.

### BACKGROUND

Medical devices, in particular implantable medical devices (implants), for providing electrical stimulation to body tissues, for monitoring physiologic conditions, and for providing alternative treatments to drugs are well known in the art. Such active or passive implantable medical devices often comprise a power source and a processor connected with an electronic circuit forming an electronic module accommodated within a hermetically sealed device housing. Leadless medical devices work without any leads separate to the housing and often comprise at least one electrical contact area at an outer surface of their housing forming a direct electrical contact to the body tissue of the patient carrying the implant.

Usually, medical devices are partially coated on the outer surface of the housing in order to achieve a better electrical function. There are different state-of-the-art types of manufacturing for such coating which are time consuming and expensive.

The known immersion bath coating method using silicones needs a curing time after the coating process of several hours. Further processing must be stopped during this time. Additionally, the rate of deficient products is high and the process needs extensive manual support. The known vacuum-based coating process using, for example, parylene is an expensive batch process, which has a high cycle time. Both known manufacturing methods are not well suitable for automation or one-piece-flow.

Accordingly, there is a desire for a robust, more cost-effective and less time consuming manufacturing method for easy automation. Accordingly, the problem consists in finding a medical device achieved with less manufacturing costs and time and high automation potential.

The present disclosure is directed toward overcoming one or more of the above-mentioned problems, though not necessarily limited to embodiments that do.

### SUMMARY

At least the above problem is solved by a method having the features of claim 1 and a medical device with the features of claim 8.

In one embodiment, a manufacturing method for a medical device having an assembly comprising an elongated solid housing with an outer surface and a maximum outer diameter, at least one electrical contact area at the outer surface of the housing and a processor encapsulated within the housing is provided. Further, a tube consisting of an insulating material, e.g., a plastic, is provided, wherein an inner diameter of the tube is greater than the maximum outer diameter of the assembly. Further, in a second step, the assembly is accommodated within the tube such that at least one electrical contact area of the assembly is not covered and a shrinking step is applied to the tube such that the shrunken tube is firmly attached to the outer surface of the housing. The shrunken tube forms the electrically insulating coating of the housing.

The maximum outer diameter is the maximum dimension of the housing measured transversely (perpendicular) to the longitudinal direction of the elongated medical device. The longitudinal direction of the medical device may be formed by the longitudinal axis of the medical device.

The above manufacturing method is a cost-effective coating process for a medical device forming an electrical insulating (non-conductive) layer in order to ensure sufficient electrical insulation and physical protection during active operation inside an environment of a human or animal body. There are advantages from the process profitability view with regard to the inventive method comprising a reduced cycle time, a better handling in the process flow, less required machinery equipment and manual support. Accordingly, the inventive manufacturing process is less time consuming and costly as well as better suitable for automation.

There is a high automation potential for the accommodation process of the assembly in the tube, for example, by using a pick-and-place treatment including a specific handling machinery. An optical inspection may further be used to judge the accommodation process. Additionally, the shrinking step can be performed at large scale in a continuous furnace process, wherein the optimized temperature profile may be generated by a hot air flow treatment. The function of the electrically insulating layer may be tested indirectly by optical inspection or directly by adapted conductivity tests.

In order to assist the accommodation process, the tube shape may be modified in the section of the tube aperture by an enlarged outer diameter or by slightly convex edges to the outside. Additionally or alternatively, instead of using one tube (element) having one material for use with one assembly, in another embodiment two or more separate tube elements may be used for one assembly. The two or more tube elements or tube sections may comprise the same or different materials in order to adapt the different requirements at different sections of the housing of the medical device. Additionally or alternatively, structures at the inner and/or outer surface of the tube may be provided, for example, protrusions or indentations in the form of strips, meshes, crosses and/or dots in order to improve the handling of the tube during the manufacturing process or to improve grip or adhesion of the shrunken tube to the outer surface of the housing of the medical device providing a firm connection. Additionally or alternatively, the one or more tube elements or sections may comprise at least one recess, for example, in order to not cover one electrical contact area at the housing which is not located at one of the ends of the medical device housing.

In one embodiment, the shrinking step is provided such that the shrunken tube hermetically seals the outer surface of the housing. A hermetically sealing shrunken tube at the outer surface of the housing can ensure a void and a defect free binding of the electrical insulation layer on the housing surface, which is ensured by constant controlling of the thermal treatment parameters.

In one embodiment, the shrinking step comprises a thermal treatment. For example, a thermal treatment in the temperature range 60° C. to 300° C. depending on the material of shrinking tube. For active medical implants tube materials with low shrink temperature, e.g., 60° C. to 110° C., are preferred due to temperature sensitive components of the medical device like a battery or an accumulator. An example is Polyefin LDPE or HDPE.

An easy realizable possibility for shrinking the tube is using a hot air flow treatment. Alternatively or additionally, the thermal treatment may be provided or supported by infrared or UV radiation using an infrared heater arrangement or UV lamp. Tubes for thermal shrinking comprise generally biocompatible thermoplastic materials, for example, polyethylene (PE), fluoropolymers like polyvinylidene fluoride (PVDF) and Fluorinated ethylene propylene (FEP) or polytetrafluoroethylene (PTFE).

In another embodiment, initially the inner diameter of the tube may be smaller than the maximum outer diameter of the assembly. In this embodiment, the manufacturing method comprises the additional step of swelling applied to the tube such that the inner diameter of the swollen tube is greater than the maximum outer diameter of the assembly prior the accommodation of the assembly within the tube, and wherein the shrinking step comprises a drying treatment. The drying treatment may be conducted at a temperature which is below the above temperature range for thermal treatment so that this process is suitable for temperature sensitive tube materials. The drying treatment releases the incorporated chemicals. The drying treatment may be performed in the form of air drying, drying within a furnace and/or microwave drying using vacuum or an atmosphere formed by at least one gas of the group comprising nitrogen, argon, air, at a temperature of room temperature to a maximum applicable temperature, e.g., for a battery based on lithium iodide. Typical long term temperatures are about 55° C.

In the above embodiment, the swelling step may be provided by a chemical treatment with a swelling fluid comprising an alkane, for example, heptane. The chemical treatment includes a particular exposure time of the tube inside the swelling fluid. This may be realized offline process as a separated preparation step or within the process flow, for example, by a full dipping of the tube into a reservoir of the swelling fluid, for example, provided by a container. Alkanes like heptane are available in large quantities. An easy handling within the process flow at room temperature is possible, which makes the material an excellent material for a cost effective manufacturing method.

There are variations of tube dimensions possible, however for the present invention the following inner diameters compared to outer housing diameter is typically 1.1:1 up to 4:1 based on shrinking ratio of the tube.

In one embodiment, the tube material deforms plastically during the shrinking step as it changes the shape irreversible after cooling down to room temperature or the temperature of the human or animal body. For example, the tube comprises material of at least one of the following groups of plastic materials comprising, e.g., PE, PCDF, FEP, PTFE.

In another embodiment of the manufacturing method, the tube comprises an adhesive at its inner surface prior the accommodation of the assembly within the tube, wherein the adhesive is activated during and/or after the shrinking step. The usage of an adhesive as an additional material supports the accommodation of the assembly within the tube, for example, in case the adhesive is liquid-like, by reducing the friction at the interface at the outer surface of the housing and the inner surface of the tube. The intermediate adhesive may be automatically thermally cured during the shrinking step leading to a tight bonding between the assembly and tube surface. For this purpose adhesives comprising at least one of the following group comprising, e.g., epoxy, silicone, polyurethane or cyanoacrylate may be used, wherein the adhesives have different material properties, for example, mechanical strength after activating, e.g., with thermal treatment and/or UV treatment.

In one embodiment, an implantable medical device is realized comprising an elongated solid housing with an outer surface, at least one electrical contact area at the outer surface of the housing, and a processor encapsulated within the housing. Additionally, a plastic and electrically insulating layer is firmly attached to the outer surface of the housing, wherein the insulating layer is obtained by shrinking a tube to the outer surface of the housing, wherein the insulating layer does not cover the at least one electrical contact area.

Different embodiments of the tube are indicated above. The medical devices or the above-defined assembly is, for example, an implantable loop recorder (subcutaneous device for monitoring cardiac activity), an implantable Cardiac Pacemaker, an Implantable Leadless Pacer (ILP), an Implantable Leadless Pressure Sensor (ILPS), an Implantable Cardioverter-Defibrillator (ICD) or a Subcutaneous Implantable Cardioverter-Defibrillator (S-ICD).

The above defined manufacturing method is easily adaptable to different shapes of medical devices used for different applications in the human or animal body. There is a good miniaturization potential if an integrated processor is used in the device. An increased flexibility of the device is provided by using different complex electronic circuit designs for different device applications.

In one embodiment of the medical device, the housing has a circular cross-section. The shape of the tube having a similar geometry as the housing, helps for the accommodation process. Further, the connection of the insulation layer and the device will become closer, more homogenous and more accurate after the shrinking or drying treatment. However, different cross section geometries of the medical device and the tube are possible, as well. In each case, it is advantageous to adapt the shape of the tube's cross section to the outer shape of the housing of the medical device.

In one embodiment of the medical device, the housing has a bag-like shape and/or the housing has a relation of length to width which is greater than 2 having, e.g., a geometry of 2 cm to 6 cm in length and 1 cm to 3 cm in width. Furthermore, cuboid shaped housings or other non-circular housings of any shape may also be applicable.

In one embodiment, the device comprises an energy supply unit encapsulated within the housing, wherein the energy supply unit is electrically connected to the processor, wherein the energy supply unit is, for example, a battery, an accumulator or a generator. With the proposed design of an energy supply unit, a processor, a housing and an insulation layer the medical device forms a self-supporting construction inside the human or animal body environment.

In one embodiment, the device may comprise an intermediate layer located between the outer surface of the housing and the electrically insulating layer, wherein the intermediate layer comprises an adhesive. The adhesive material improves the adhesive strength of the insulating layer and the housing of the medical device. Additionally, a layered stack, i.e., the insulating layer, the adhesive film, and the layer of the housing material, may form better diffusion barrier properties against the human or animal body environment. This increases the lifetime of the medical device against electro migration.

In one embodiment, the medical device is an active implant. Active implants are able to fulfill complex tasks and may include an electronic and/or an intelligent unit.

In one embodiment, the material of the electrically insulating layer comprises at least one material of the group comprising silicone and parylene. Parylene as an insulation layer is known, on the one hand, to be relatively robust against harsh environmental impacts and forms very homogenous coating layers. On the other hand, it is a biocompatible material without containing any solvents or softening agents. Silicone is a biocompatible material which has the ability to fill the smallest gaps homogenously.

In one embodiment, the material of the housing comprises a hermetically sealing material, e.g., metal. The material of the at least one electrical contact area comprises a biocompatible and electrically conductive, e.g., a metal like titanium.

In one embodiment, the processor of the device is adapted to receive electrical signals, wherein the signals may be measured at the least on one electrical contact area, and/or to process electrical signals and/or to send electrical signals. The processor may comprise a sensor, which measures and/or processes all relevant signals and information data from the human or animal body environment, and/or a sender, wherein the signals and information data are transmitted to an outside receiver. Such receiver may be contained in a mobile device containing, for example, a medical application of an attending healthcare professional, patient and/or hospital. Further, a receiver and/or sender unit of, e.g., a mobile device, a computer or another medical device located outside the body may communicate with the device accommodated inside the body.

Additionally, the processer may analyze the incoming signals and process them using the received information data. As a result, parameters of the device settings may be modified for a better adaption to the patient's needs. The processor may be integrated in an electronic circuit comprising the energy supply unit as voltage source. Further, the at least one electrical contact area with additional resistive, capacitive and inductive elements mounted on a substrate formed by a PCB, IMS, LTCC, thin film, thick film, DBC and AMB. The processor may comprise at least one element of the group comprising an integrated circuit, micro controller, p-n diode, transistors, MOSFET and IGBT depending on the medical device complexity. There are different types of senders may be used such as a transceiver, transmitter, transponder, emitter, ultrasonic emitter and RFID transponder.

A full and enabling disclosure of the present invention, including the best mode thereof, directed to one of ordinary skill in the art is set forth in the following specification. Thereby, further features and advantages are presented that are part of the present invention independently of the features mentioned in the claims.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The specification refers to the accompanying figures showing schematically:

FIG. 1 a first step of a first embodiment of an inventive manufacturing method with a medical device in a side view, and

FIG. 2 a tube in a longitudinal section,

FIG. 3 a second and third step of the first embodiment of the manufacturing method with the medical device in a side view and the tube in a longitudinal section,

FIG. 4 the result of the third step shown in FIG. 3,

FIG. 5 an initial tube of a second embodiment of an inventive manufacturing method in a longitudinal section,

FIG. 6 the tube of FIG. 5 after a manufacturing step of the second embodiment in a longitudinal section, and

FIG. 7 the medical device of FIG. 1 in a front view.

### DETAILED DESCRIPTION

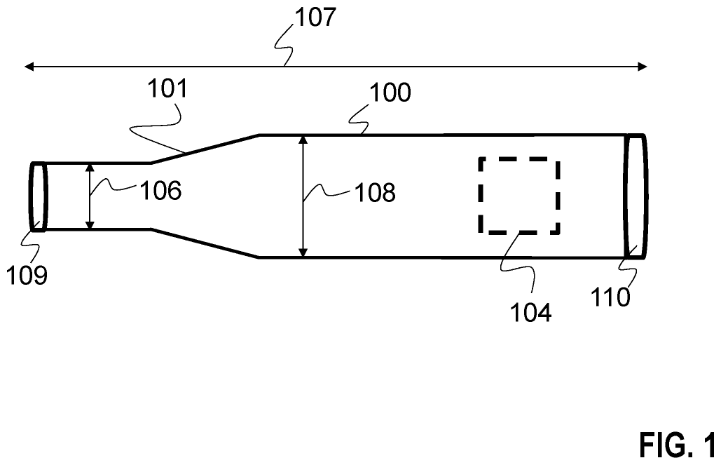

A medical device 100 is shown in FIG. 1, for example, an implantable loop recorder or an implantable leadless pacer, which comprises a solid housing 101 with a first contact area 109 and a second contact area 110 located at the proximal and the distal end of the housing 101, respectively. The medical device 100 according to FIG. 1 has a length 107 and a width 108, wherein the width 108 forms at the same time the maximum outer diameter of housing 101 and the first diameter of a distal section of the housing 101. Further, the housing 101 comprises a second diameter 106 at a proximal section of the housing 101. The housing 101 further comprises an intermediate section between the proximal section with the smaller diameter 106 and the distal section with the greater diameter (width 108).

Additionally, the medical device 100 comprises an encapsulated processer 104, a battery (not shown) forming and being part of an electronic circuit (not shown) provided on a circuit board (e.g., printed circuit board, PCB). The electronic circuit is electrically connected to the first and second contact area 109, 110, which are in contact to the human body environment if the medical device 100 is implanted in a human body. Additionally, the processor 104 may comprise a receiver and/or a sender (both not shown).

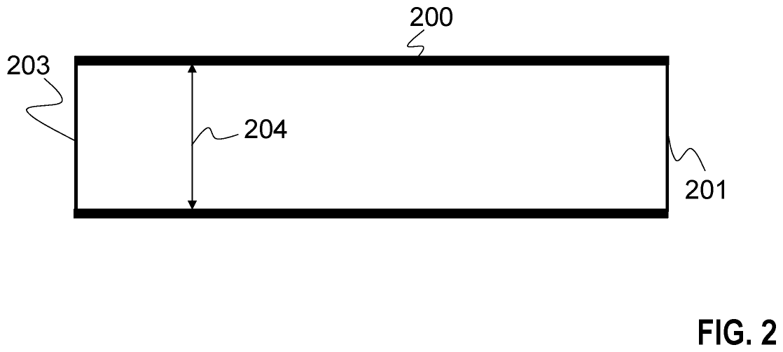

FIG. 2 shows a tube 200 for the electrically insulating layer with a first aperture 201 and a second aperture 203. The tube 200 has an inner diameter 204, which is relevant for the accommodation of the medical device 100 and shrinking described below.

After providing the medical device 100 and the tube 200, the medical device 100 is placed within the tube 200 which is possible since the inner diameter 204 of the tube 200 is greater than the maximum outer diameter (width 108) of the medical device 100. The medical device 100 is introduced into the tube 200 through the first aperture 201 or the second aperture 203 of the tube and accommodated such that the first and second contact area 109, 110 is not covered.

Before accommodating the medical device 100 within tube 200, an adhesive may additionally be applied at the inner surface of the tube 200 or on the surface of the device 100 thereby reducing the friction at the interface of the device 100 and tube 200.

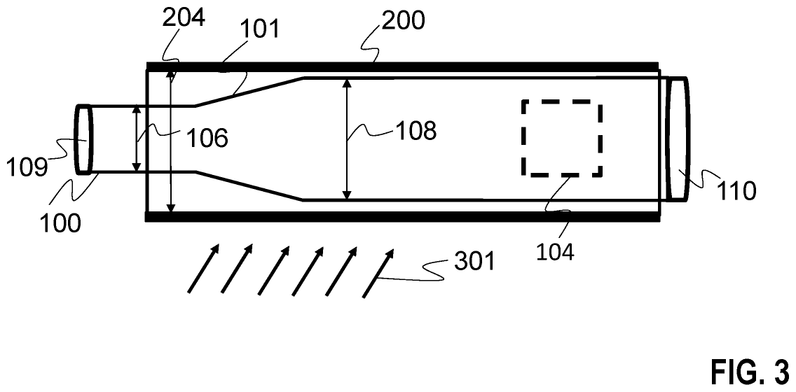

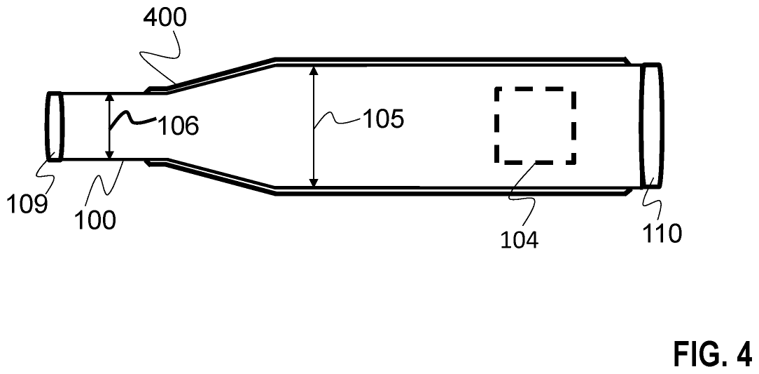

After correct accommodation of the medical device 100 within the tube 200, a thermal treatment is applied, e.g., with the following parameters 80° C. for 30 seconds. The thermal treatment is symbolized by arrows 301 in FIG. 3 and leads to shrinking of the tube 200 such that it is firmly attached to the outer surface of the housing 101 at the end of this process step. FIG. 4 depicts the result of the thermal treatment showing an electrically insulating layer 400 formed by the shrunken tube 200 at the outer surface of the housing 101 of the medical device 100, wherein the electrically insulating layer 400 does not cover the first and second contact area 109, 110.

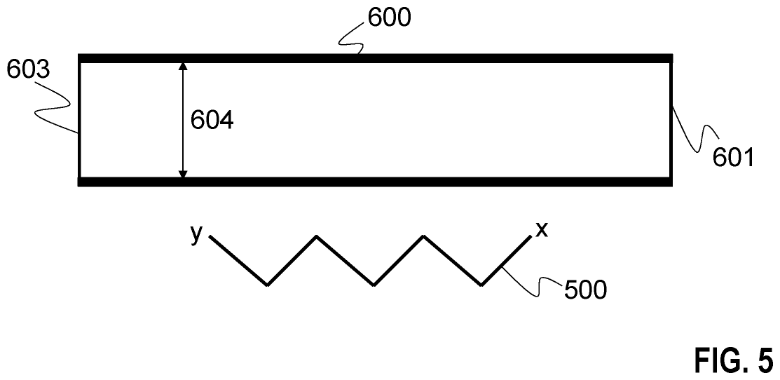

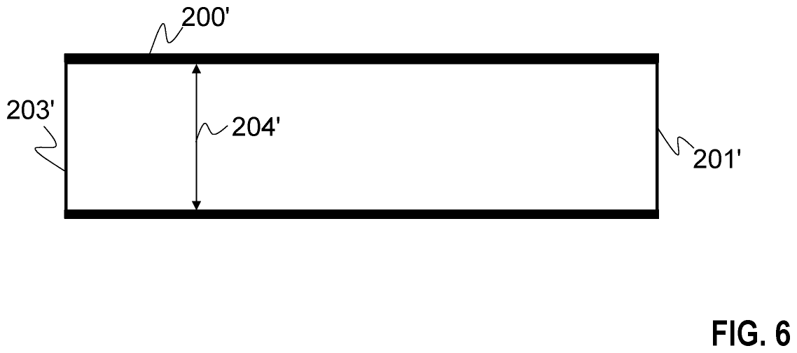

In an alternative embodiment, as an initial element a tube 600 may be used having an initial inner diameter 604 which is smaller than the maximum outer diameter (width 108) of the medical device 100. Such tube 600 is shown in FIG. 5. The tube 600 is swollen by immersion within heptane (see symbol 500) thereby increasing the dimensions of the tube 600 resulting in the tube 200′ with an increased inner diameter 204′ as shown in FIG. 6. The swollen tube 200′ is used with the manufacturing method shown in FIGS. 1 to 4 and described above, wherein the thermal treatment may be replaced or supplemented by a drying treatment in order to shrink the swollen tube 200′. In the drying treatment, the following process parameters may be applied 35° C.

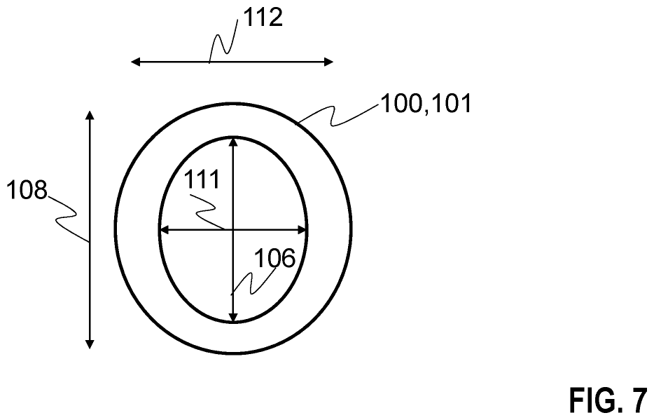

FIG. 7 depicts a front view of the medical device shown in FIG. 1. The non-circular (oval) cross section of the proximal section of the housing 101 has second diameter 111 and the oval cross section of the distal section of the housing 101 has second diameter 112, as well. The medical devices implantable loop recorder, implantable leadless pacer, implantable leadless pressure sensor, (subcutaneous) Implantable Cardiac Defibrillator ((s)ICD) have different shapes and dimensions resulting in different geometric requirements for the tube 200 to be shrunken at the outer surface of housing 101 for firm attachment.

As indicated above, the inventive manufacturing method creates, and the inventive medical device comprises, a partial electrical insulation layer 400 which may easily be adapted in an automated process and to different shapes of the medical device. The insulation layer may enhance the biocompatibility of the outer surface of the medical device.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE NUMBERS

100 medical device
101 housing
104 processor
106 diameter
107 length
108 width, maximum outer diameter
109 first contact area
110 second contact area
111, 112 second diameter
200, 200′ tube
201, 201′ first aperture
203, 203′ second aperture
204, 204′ inner diameter
301 arrows
400 insulating layer
500 heptane
600 initial tube
601 first aperture
603 second aperture
604 inner diameter

1. A manufacturing method for a medical device having an assembly comprising
an elongated solid housing with an outer surface and a maximum outer diameter,
at least one electrical contact area at the outer surface of the housing, and
a processor within the housing,
wherein the method comprises the following steps:
providing the assembly and a tube consisting of an electrically insulating material, wherein an inner diameter of the tube is greater than the maximum outer diameter of the assembly,
accommodating the assembly within the tube such that at least one electrical contact area of the assembly is not covered, and
applying a shrinking step to the tube such that the shrunken tube is firmly attached to the outer surface of the housing.

2. The method of claim 1, wherein the material of the tube comprises a polymer material.

3. The method of claim 1, wherein the shrinking step comprises a thermal treatment and/or infrared and/or UV treatment.

4. The method of claim 1, wherein the shrinking step is provided such that the shrunken tube hermetically seals the outer surface of the housing.

5. The method of claim 1, wherein initially the inner diameter of the tube is smaller than the maximum outer diameter of the assembly, wherein the method comprises the additional step of swelling applied to the tube such that the inner diameter of the swollen tube greater than the maximum outer diameter of the assembly prior the accommodation of the assembly within the tube, and wherein the shrinking step comprises a drying treatment.

6. The method of claim 5, wherein the swelling is provided by a chemical treatment with a fluid comprising an alkane, for example heptane.

7. The method of claim 1, wherein the tube comprises an adhesive at its inner surface prior the accommodation of the assembly within the tube, wherein the adhesive is activated during or after the shrinking step.

8. A medical device comprising:
an elongated solid housing with an outer surface, at least one electrical contact area at the outer surface of the housing, and a processor encapsulated within the housing, and
a plastic and electrically insulating layer firmly attached to the outer surface of the housing, wherein the layer is obtained by shrinking a tube to the outer surface of the housing, wherein the insulating layer does not cover the at least one electrical contact area.

9. The medical device of claim 8, wherein the housing has a circular cross-section.

10. The medical device of claim 8, wherein the housing has a bag-like shape and/or the housing has a relation of length to width which is greater than 2.

11. The medical device of claim 8, wherein the device comprises an energy supply unit encapsulated within the housing, wherein the energy supply unit is in electrical contact with the processor, wherein the energy supply unit is, for example, a battery, an accumulator or a generator.

12. The medical device of claim 8, wherein the device comprises an intermediate layer located between the outer surface of the housing and the electrically insulating layer, wherein the intermediate layer comprises an adhesive.

13. The medical device of claim 8, wherein the medical device is one of an implantable loop recorder, an implantable cardiac pacemaker, an implantable leadless pacemaker, an implantable leadless pressure sensor, an implantable cardioverter-defibrillator or a subcutaneous implantable cardioverter-defibrillator.

14. The medical device of claim 8, wherein the material of the electrically insulating layer comprises at least one material of the group comprising silicone and parylene.

15. The medical device of claim 8, wherein the processor is adapted to receive electrical signals, wherein the signals are preferably measured at least one contact area, and/or to process electrical signals and/or to send electrical signals.
